(19) United States
(12) Patent Application Publication
(10) Pub. No.: US 2009/0198200 A1
(43) Pub. Date: Aug. 6, 2009

(54) WOUND DRESSING HAVING UNDERCUT CHANNELS FOR NEGATIVE PRESSURE WOUND THERAPY

(76) Inventors: David Tumey, Germantown, MD (US); Richard C. Vogel, Potomac, MD (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/357,733

(22) Filed: Jan. 22, 2009

Related U.S. Application Data
(60) Provisional application No. 61/022,574, filed on Jan. 22, 2008.

(51) Int. Cl. A61F 13/00

(52) U.S. Cl. 604/305

(57) ABSTRACT

A wound dressing defines a channel. The channel includes a body portion having a width and a neck portion having a width less than the width of the body portion.

### CROSS REFERENCES TO RELATED CASES

This application claims priority to and the benefit of U.S. Provisional Patent Application Ser. No. 61/022,574, filed Jan. 22, 2008, which is incorporated herein by reference in its entirety.

This application is related to U.S. patent application Ser. No. 11/350,089, filed Feb. 9, 2006, which is a continuation-in-part of U.S. patent application Ser. No. 11/237,880, filed Sep. 29, 2005, which is a continuation of U.S. patent application Ser. No. 11/198,148, filed Aug. 8, 2005, entitled “Wound Irrigation Device,” each of which is incorporated herein by reference in their entirety.

### BACKGROUND

The invention generally relates to dressings for use in healing wounds through Negative Pressure Wound Therapy (NPWT) applications.

In NPWT, a vacuum source is connected to a semi-occluded or occluded wound dressing. Various porous dressings comprising gauze, felts, foams, beads and/or fibers can be used in conjunction with an occlusive semi-permeable cover and a controlled vacuum source. NPWT is also known as vacuum drainage or closed-suction drainage. In addition to using negative pressure wound therapy, many devices employ concomitant wound irrigation.

NPWT dressings are often manufactured in rectangular or oval shapes, and provided in a size greater than the wound to be treated. Clinically, the caregiver (or medical practitioner) typically cuts the dressing to a size and shape to fit the dressing in the wound in a way that does not cause compression (and subsequent reduction in porosity) of the sponge after it is placed in the wound.

Once the dressing is placed in the wound and the bottom surface abuts the wound bed, tubing is affixed to the dressing to permit fluid communication between the tubing and the dressing to provide irrigation and/or suction drainage. The tubes are typically held in place while the caregiver applies a covering over the woundsite to seal the dressing and tubing in place (e.g., with a fluid-tight seal) so that sub-atmospheric pressures can be maintained at the woundsite throughout the therapy. Typically, a single caregiver will apply the dressing to the patient's wound, making it difficult to hold the tubing in place by hand, while also manipulating the covering to seal the wound.

One possible solution includes placing the tubing on the bottom surface of the dressing. This, however, may cause necrosis of the tissue in the wound bed directly below the tubing and may cause damage to delicate underlying wound structure and potentially rupture blood vessels. Another possible solution includes affixing the tubing to the top of the dressing using surgical or other adhesive tape. This placement causes the tubing to sit above the dressing's top surface, which may cause the covering to block the opening of the tube. Additionally, the adhesive tape may block the opening of the tube.

Affixing the tubing to the skin adjacent the wound using surgical or other adhesive tape also is inadequate. The distal ends of the tubes are free to move across the surface of the dressing making it difficult for the caregiver to apply the dressing, the tubing is still located above the dressing's top surface, the adhesive tape might block the opening of the tube, and the covering might trap the adhesive tape between itself and the skin adjacent the wound bed, potentially causing tissue macerations.

Thus, a need exists for a dressing that maintains fluid communication with suction/irrigation tubing during application and operation of a NPWT system on a patient, without harming the patient. Additionally, a need exists for a dressing that allows easy repositioning of suction/irrigation tubing in fluid communication with the dressing.

### SUMMARY

A wound dressing defines a channel. The channel includes a body portion having a width and a neck portion having a width less than the width of the body portion.

### BRIEF DESCRIPTION OF THE DRAWINGS

FIG. 1 shows a Negative Pressure Wound Therapy (NPWT) system with irrigation, according to an embodiment.

FIG. 2 is a perspective view of a dressing with undercut channels for use in NPWT, according to an embodiment.

FIG. 3 is a front view of the dressing of FIG. 2.

FIG. 4 is an exploded view of a portion of a NPWT system including the dressing of FIG. 2, according to an embodiment.

FIG. 5 shows the dressing of FIG. 2 applied to a wound on the leg of a patient.

FIG. 6 is a side view of tubing for use in NPWT with fenestrations at one end portion, according to an embodiment.

FIG. 7 is a side view of tubing for use in NPWT without fenestrations, according to an embodiment.

FIG. 8 is a front view of a dressing with square-shaped undercut channels for use in NPWT, according to an embodiment.

FIG. 9 is a front view of a dressing with trapezoidal-shaped undercut channels for use in NPWT, according to an embodiment.

FIG. 10 is a side view of tubing disposed within a triangular tube housing for use in NPWT, according to an embodiment.

FIG. 11 is a front view of a dressing for use with the tubing of FIG. 10, according to an embodiment.

FIG. 12 is a top view of a dressing for use in NPWT having closed neck undercut channels, according to an embodiment.

FIG. 13 is a front view of a dressing for use in NPWT having closed neck undercut channels, according to an embodiment.

FIG. 14 is a front view of a dressing for use in NPWT having closed neck undercut channels for use with the tubing and tube housing of FIG. 10, according to an embodiment.

FIG. 15 is a top view of a dressing for use in NPWT with undercut channels having a perforated layer covering the dressing, according to an embodiment.

FIG. 16 is a side view of a dressing for use in NPWT with undercut channels having a perforated layer covering the dressing, according to an embodiment.

FIG. 17 is a top view of a dressing for use in NPWT with undercut channels having a removable layer of foam in the neck of the undercut channels, according to an embodiment.

FIG. 18 is a side view of a dressing with undercut channels having a removable layer of foam in the neck of the undercut channels, according to an embodiment.

### DETAILED DESCRIPTION

Various embodiments generally relate to wound dressings used in Negative Pressure Wound Therapy (NPWT). According to an embodiment, undercut channels in a wound dressing are used to moveably secure suction/irrigation tubing to the dressing. Undercut channels are channels cut into the dressing configured to receive tubing. The channels can be any size or shape. The tubing can be held in the undercut channels by friction and may be readily repositioned if necessary.

As used herein, the terms proximal portion or proximal end refer to the portion or end, respectively, of a device that is closest to a medical practitioner (e.g., a physician) when performing a medical procedure, and the terms distal portion or distal end refer to the portion or end, respectively, of the device that is furthest from the physician during a medical procedure. For example, a distal end or portion of a suction/irrigation tube as described herein refers to the end or portion of the tube that is connected to the wound dressing. The proximal end or portion is the end or portion of the tube that is connected to a suction source or an irrigation source.

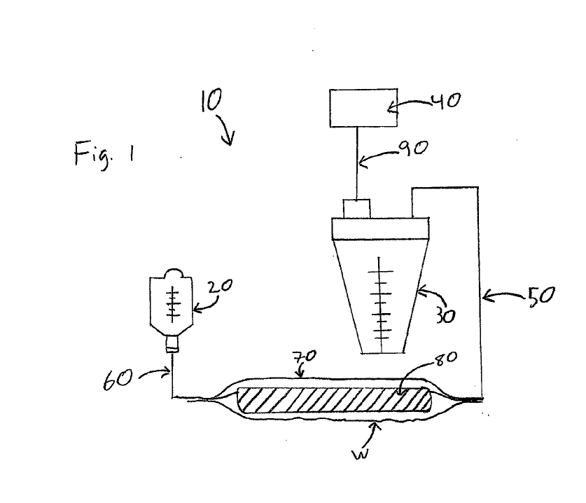

FIG. 1 shows a NPWT system with wound irrigation, according to an embodiment. The NPWT system 10 has a wound dressing 80, which is placed in a wound W. The distal end portions of suction tube 50 and irrigation tube 60 are connected to the wound dressing 80 in one of the manners described in greater detail below. A covering 70, such as a semi-permeable occlusive sheet or drape, covers the wound dressing 80. The covering 70, for example, can be made of polyurethane film such as that available under the trademark Tegaderm™. The covering 70 is sealed to the skin surrounding the wound by, for example, an adhesive. The proximal end portion of the suction tube 50 is connected to a fluid collection canister 30. The fluid collection canister 30 is connected to a suction source 40 by tubing 90. The proximal end portion of the irrigation tube 60 is connected to a reservoir 20 that contains a solution, such as, by way of example, an aqueous topical antibiotic solution, isotonic saline, Dakin's solution, or a Sulfamide Acetate solution, for use in providing therapy to the wound W.

When the suction source 40 is turned on, a negative pressure is produced at the wound W and fluid from the wound dressing 80 travels through the suction tube 50 and is collected in the fluid collection canister 30. The negative pressure at the wound dressing 80 and/or gravity can cause the solution contained in the reservoir 20 to travel through the irrigation tube 60 and into the wound dressing 80.

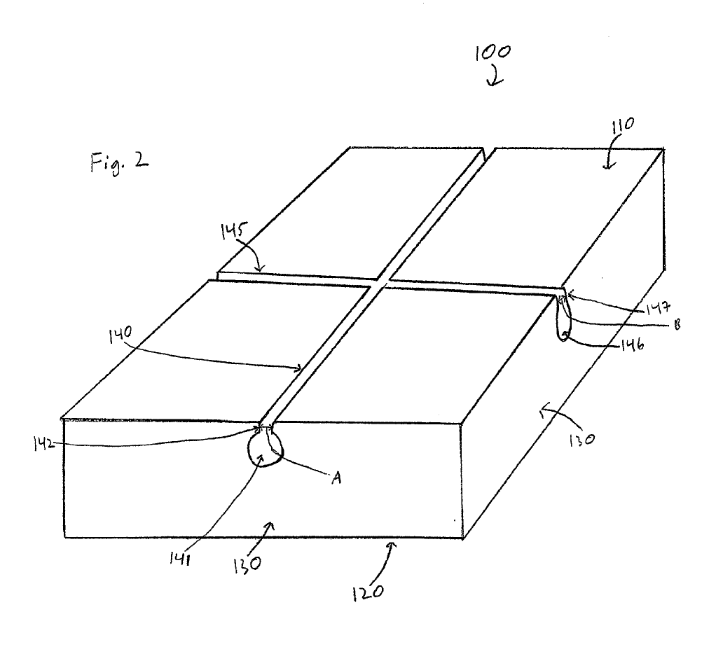

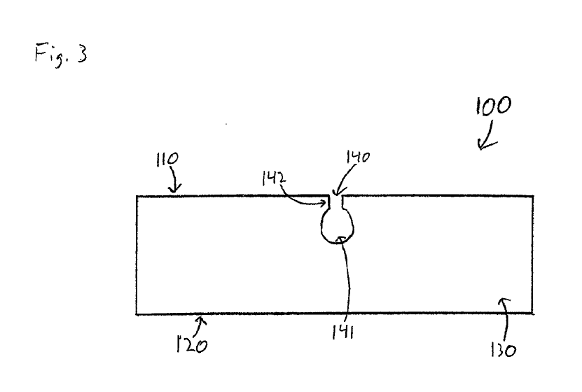

FIG. 2 and FIG. 3 show one embodiment of a dressing 100 for use in NPWT. The dressing 100, for example, can be manufactured from polyurethane foam, polyvinyl alcohol foam, felt or other suitable porous material. The dressing 100 has a top surface 110, a bottom surface 120 and side surfaces 130. The top surface 110 includes undercut channels 140, 145. The undercut channels 140, 145 are configured to receive tubing for use in NPWT. The undercut channels 140 and 145 can be produced and/or manufactured using myriad methods and/or using myriad devices such as the methods and devices shown and described in U.S. Provisional Patent Application No. 61/023,998, filed Jan. 28, 2007 entitled “Method and Apparatus for Manufacturing Wound Dressing for Negative Pressure Wound Therapy” which is incorporated herein by reference in its entirety. For example, the undercut channels 140, 145 cab be manufactured using, for example, a hot-wire technique, a wire saw, or a die-cutting technique.

The undercut channels 140, 145 of FIG. 2 include body portions 141, 146 and neck portions 142, 147. In this embodiment, the body portions 141, 146 are circular in shape. The body portions 141, 146 have a respective circumference (or perimeter or diameter) that is substantially equal to (e.g., slightly smaller than) a circumference (or perimeter or diameter) of a tubing to be placed in the undercut channels 140, 145. This allows the tubing to fit snuggly into the body portions 141, 146 of the undercut channels 140, 145. The neck portions 142, 147 of the undercut channels 140, 145 have a width A, B less than the diameter of the tubing to be placed in the undercut channels 140, 145.

While placing the tubing into the undercut channels 140 or 145, the tubing will be able to pass through the neck portion 142 or 147 to the body portion 141 or 146, respectively due to the flexibility of the foam near the neck portions 142 or 147. Once in the body portion 141 or 146, the tubing will be unable to escape through the neck portion 142 or 147 during normal use because the width A of the neck portion 142 or 147 is less than the circumference of the tubing and the neck portion 142 or 147 has sufficient rigidity to maintain the position of the tubing. The tubing, however, can be removed by a caregiver pulling the tubing through the neck portion 142 or 147. Accordingly, the tubing is movably secured within the body portion 141, 146 of the undercut channel 140, 145 and a caregiver can easily reposition the tubing if necessary. While the embodiment illustrated in FIG. 2 and FIG. 3 has two undercut channels 140, 145, any number of undercut channels may be used to attach tubing to the dressing 100.

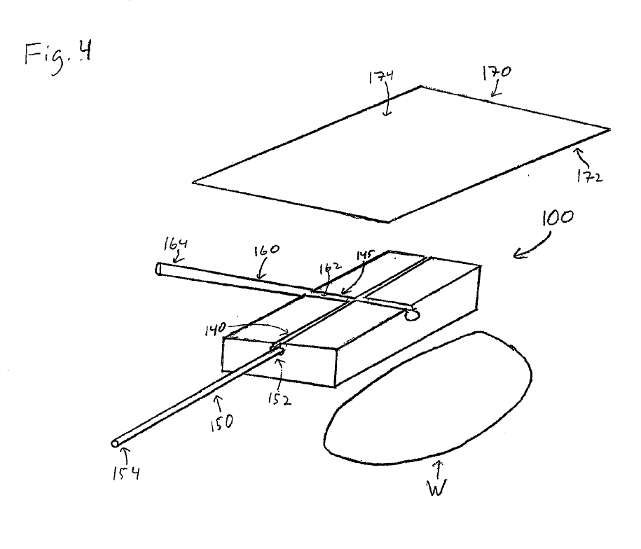

FIG. 4 is an exploded view of a portion of a NPWT system including the dressing of FIG. 2, according to an embodiment. The dressing 100 can be cut to fit the wound of a patient W. A suction tube 150 has a distal end portion 152 and a proximal end portion 154. The distal end portion 152 of the suction tube 150 is placed in an undercut channel 140 in the dressing 100. The proximal end portion 154 of the suction tube 150 is connected to a suction source (not shown in FIG. 4).

An optional irrigation tube 160 is attached to the dressing 100 by placing a distal end portion 162 of the irrigation tube 160 in a second undercut channel 145. A proximal end portion 164 of the irrigation tube 160 is attached to an irrigation source (not shown in FIG. 4) to supply fluid, such as, by way of example, an aqueous topical antibiotic solution, isotonic saline, Dakin's solution, or a Sulfamide Acetate solution, to the wound W. If necessary, the suction tube 150 and the optional irrigation tube 160, which are each held in the undercut channels 140 and 145 respectively by a friction fit, can readily be repositioned.

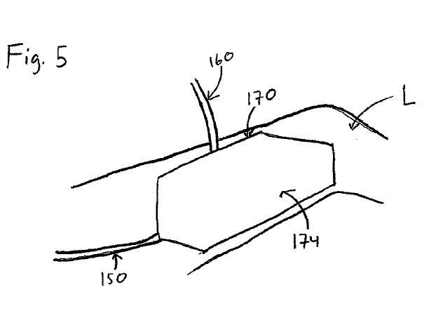

To maintain a negative pressure at the wound 120, a covering 170 such as a semi-permeable occlusive sheet or drape is applied to the dressing 100. The covering 170, for example, can be made of polyurethane film such as that available under the trademark Tegaderm™. The covering 170 has a top surface 174 and a bottom surface 172. The bottom surface 172 can contain an adhesive to seal the covering 170 to the skin surrounding the wound 120. The undercut channels 140, 145 hold the suction tube 150 and optional irrigation tube 160 in place while the covering 170 is applied to the dressing 100. Sealing the covering 170 to the skin surrounding the wound W while providing suction via the suction tube 150 provides negative pressure at the wound W. The undercut channels 140, 145 also hold the suction tube 150 and optional irrigation tube 160 in place, respectively, while administering suction and optional irrigation. FIG. 5 illustrates the dressing 100 included with a NPWT system applied to a wound on the leg of a person L.

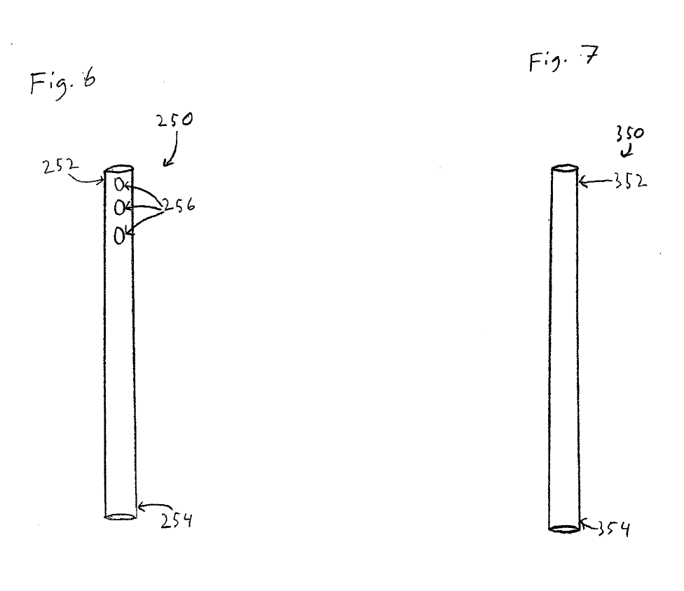

FIG. 6 and FIG. 7 each show an example of a tubing configured to be used in a NPWT system, according to embodiments. Tubing 250, shown in FIG. 6, has a distal end portion 252, configured to attach to a wound dressing such as the dressing 100 shown in FIG. 2 and FIG. 3. Tubing 250 also has a proximal end portion 254 configured to attach to a suction or irrigation source. The tubing 250 also has fenestrations 256 at the distal end portion 252. The fenestrations 256 allow for increased suction/irrigation flow from/to the dressing respectively. The fenestrations can be produced or defined using various methods such as, for example, a hole punch or a laser.

Tube 350, shown in FIG. 7, has a distal end portion 352, configured to attach to a wound dressing such as the dressing 100 shown in FIG. 2 and FIG. 3. Tube 350 also has a proximal end portion 354 configured to attach to a suction or irrigation source. Unlike tubing 250 shown in FIG. 6, tubing 350 does not contain fenestrations. The distal end portion 352 of the tubing 350 can be cut at an angle, for example 45 degrees, to help prevent the end of the tube from becoming occluded during NPWT.

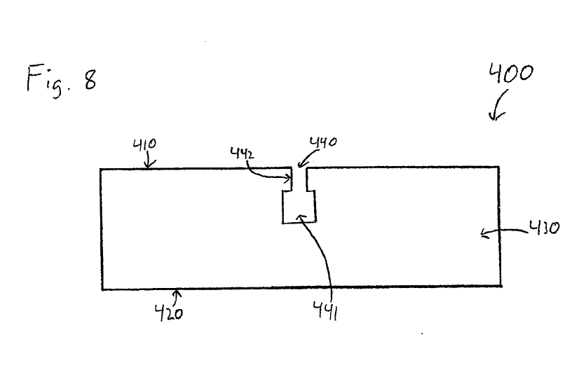

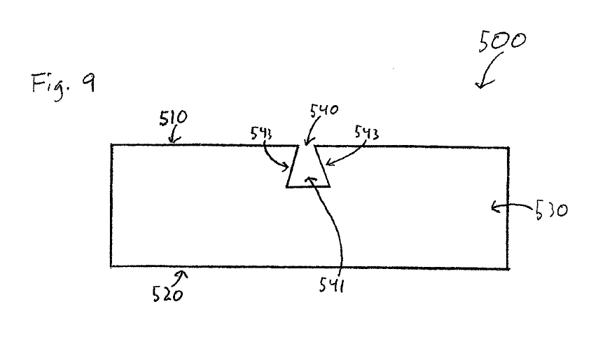

The undercut channel of a dressing can be a variety of shapes and sizes. In one embodiment illustrated in FIG. 8, the undercut channel 440 of a dressing 400 has a portion with a square-shaped cross section. Dressing 400 contains a top surface 410, a bottom surface 420 and side surfaces 430. Undercut channel 440 is formed by cutting into the top surface 410 of the dressing 400 or the side surface 430 of the dressing 400. Similar to the dressing in FIG. 2 and FIG. 3, the undercut channel 440 includes a body portion 441 and a neck portion 442.

FIG. 9 shows a dressing 500 with a trapezoidal-shaped undercut channel 540 according to an embodiment. The dressing 500 has a top surface 510, a bottom surface 520 and side surfaces 530 with the undercut channel 540 formed by cutting into the top surface 510 or the side surface 530 of the dressing 500. Rather than a neck, the body 541 has angled walls 543 which function much the same as a neck.

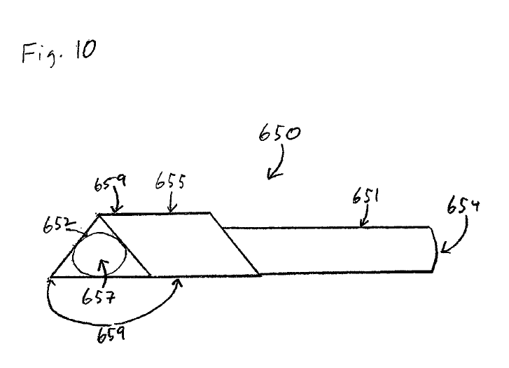

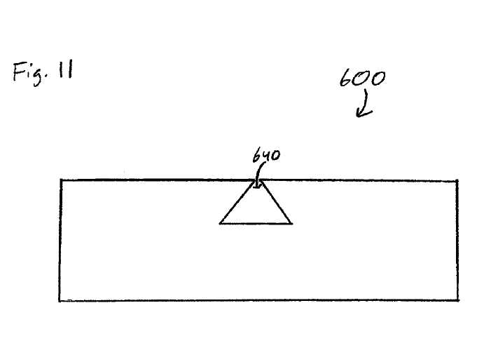

FIG. 10 shows a tube system according to another embodiment. FIG. 11 shows a dressing according to another embodiment. Tube system 650 includes a tube 651 and a tube housing 655 that can be removably coupled to the tube 651. Tube 651 has a distal end portion 652 and a proximal end portion 654. The tube housing 655 defines a lumen 657 configured to receive the distal end portion 652 of the tube 651. The proximal end portion 654 of the tube 651 is configured to connect to a suction source or an irrigation source. The tube housing 655 is triangularly shaped having points 659 and configured to be inserted into a triangularly-shaped undercut channel 640 (see FIG. 11). Matching the shape of the tube housing 655 with the shape of the undercut channel 640 can increase the strength of the friction fit between the tubing 651 and the undercut channel 640. Although a triangularly-shaped tube housing 655 and undercut channel 640 has been described, other shapes, such as a square, may be used for tube housings to match similarly shaped undercut channels.

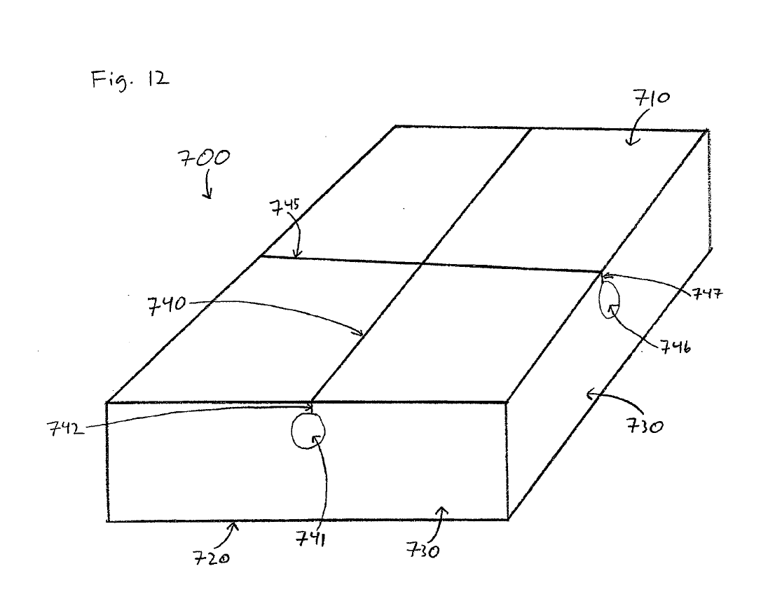

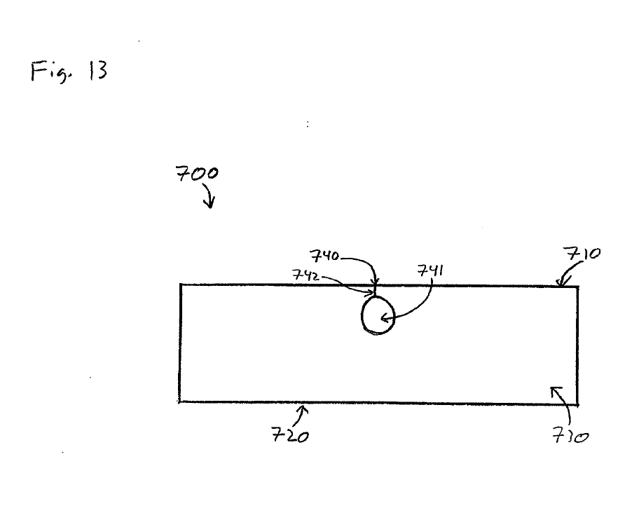

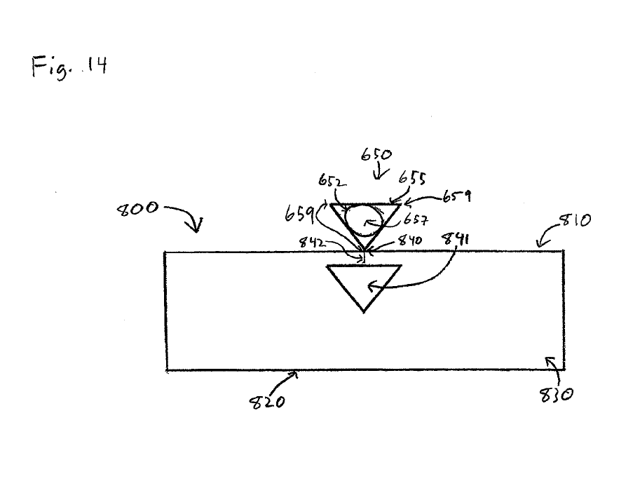

FIG. 12 and FIG. 13 show another embodiment of a dressing 700 for use in NPWT. Similar to the other dressings previously described, the dressing 700 can be manufactured from polyurethane foam, polyvinyl alcohol foam, felt or other suitable porous material. The dressing 700 has a top surface 710, a bottom surface 720 and side surfaces 730. The top surface 710 includes undercut channels 740, 745. The undercut channels 740, 745 are configured to receive tubing for use in NPWT.

The undercut channels 740, 745 of the dressing 700, include body portions 741, 746 and neck portions 742, 747. In this embodiment, the body portions 741, 746 are circular in shape. Unlike the previous discussed embodiments, however, the neck portions 742, 747 of the undercut channels 742, 747 are closed. Both sides of the respective neck portions 742 and 747, are in removable contact with each other. In other words, the neck portions 742, 747 define a vertical slot configured to allow the tubes to pass into the body portions 741, 746 of the undercut channels 740, 745. Closed neck portions 742, 747 can maintain the position of the tubes while making the tubes less likely to accidentally fall out of the undercut channels 740, 745.

FIG. 14 is a side view of an embodiment of a dressing with an undercut channel 840 having a triangular-shaped body portion 841 and a closed neck portion 842. Dressing 800 has a top surface 810, a bottom surface 820 and side surfaces 830. The undercut channel 841 is near the top surface 810. Dressing 800 is configured to be used with, for example, the tube system 650 shown in FIG. 10. The tube housing 655 is shaped to fit into the triangular-shaped body portion 841. Points 659 of the tube housing 655 can be used to move the tubing 652 through the closed neck portion 842 and into the body portion 841 of the undercut channel 840.

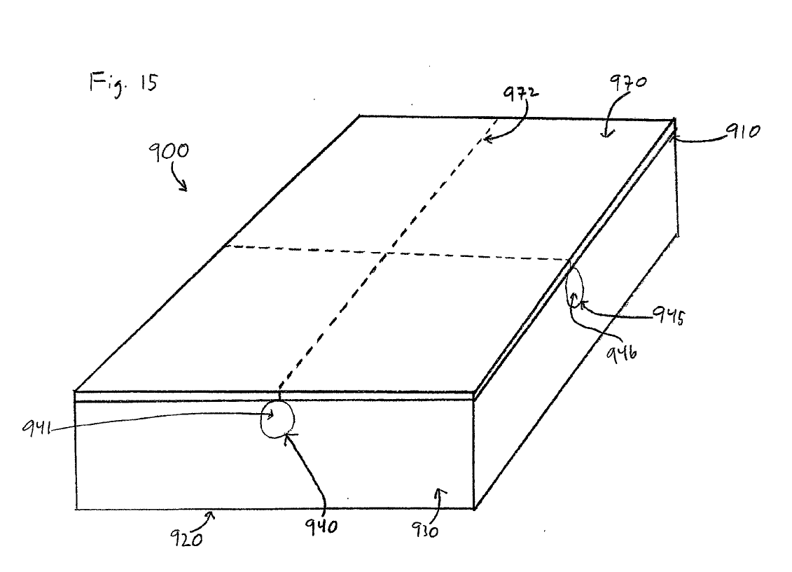

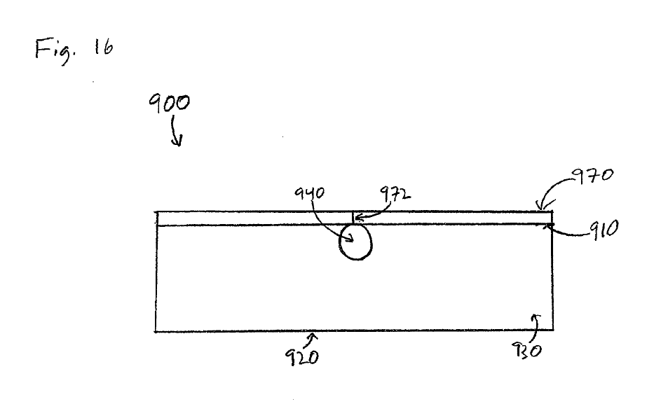

FIG. 15 is a top view of a dressing for use in NPWT with undercut channels 940, 945 having a perforated covering 970 over the dressing 900, according to an embodiment. FIG. 16 is a side view of the dressing shown in FIG. 15. Dressing 900 has a top surface 910, a bottom surface 920 and side surfaces 930. The top surface 910 includes undercut channels 940, 945. The undercut channels 940, 945 differ from other embodiments because they have a circular body portion 941, 946 and substantially no neck portion. The covering 970, which covers the top surface 910, has perforations 972 disposed above the undercut channels 940, 945. The covering 970 can be formed, for example, from foam, plastic or water-resistant paper.

Additionally, the covering 970 can be made of a material that promotes the even distribution of irrigation fluid via capillary action. Thus, in addition to holding the tubing in place, the dressing covering 970 can help ensure even distribution of therapeutic fluids throughout the dressing. The covering 970 can be attached to the dressing 900 by, for example, solvent welding, thermal bonding or pressure sensitive adhesives.

When inserting a tube in an undercut channel 940 or 945, a caregiver can first break the perforations 972 in the covering 970 to access the undercut channel 940 or 945. The perforations can be broken, for example, by the caregiver's finger or by the tip 659 of a tube housing 650 such as the one shown in FIG. 10. Once broken, the perforations 972 allow access to the undercut channels 940, 945 much like a dressing having undercut channels with a closed neck, such as dressing 700 as shown in FIG. 12 and FIG. 13.

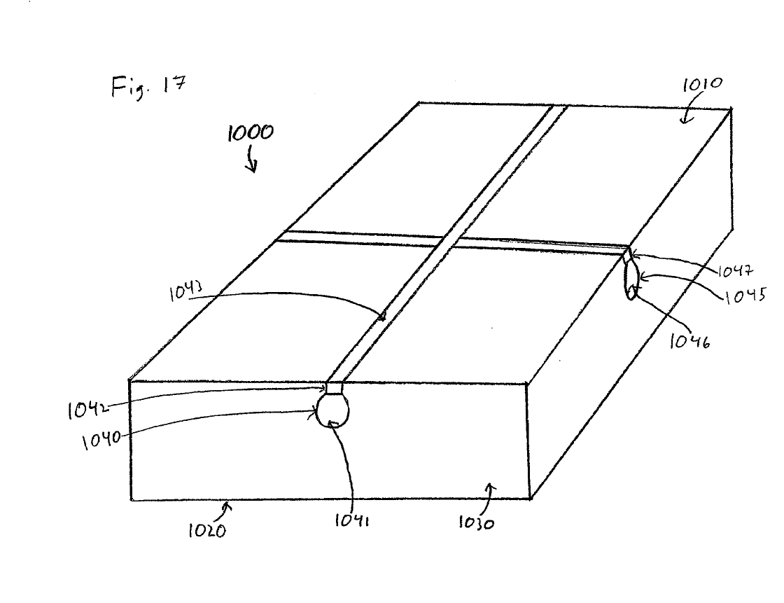

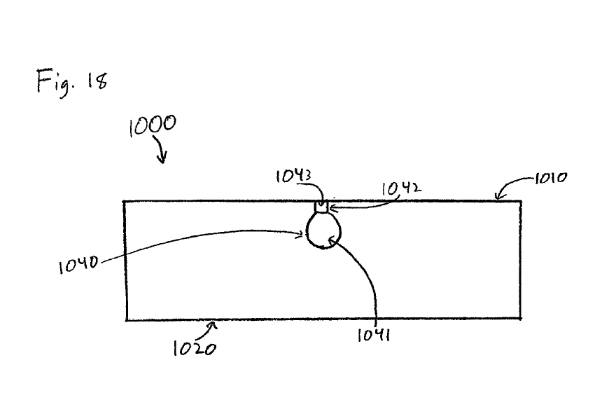

FIG. 17 and FIG. 18 show a dressing 1000 for use in NPWT having undercut channels with filled neck portions 1042, 1047. Dressing 1000 has a top surface 1010, a bottom surface 1020 and side surfaces 1030. The top surface 1010 includes undercut channels 1040, 1045. The undercut channels 1040, 1045 differ from those of above-described embodiments because neck portions 1042, 1047 are filled with a removable strip of material 1043. The removable strip of material 1043 can be constructed, for example, of the same material as the dressing or of a different material.

To insert the tubing into the body portion 1041, 1046 of the undercut channel 1040, 1045, the caregiver removes the removable strip of material 1043 from the neck portion 1042, 1047 of the undercut channel 1040, 1045 and places the tubing into the body portion 1041, 1046. Once the tubing has been placed, the removable strip of material 1043 may be returned into the neck portion 1042, 1047 of the undercut channel 1040, 1045.

While various embodiments have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular features and/or combinations of components, other embodiments are possible having any combination or sub-combination of any features and/or components from any of embodiments as described herein. Thus, the breadth and scope of the invention should not be limited by any of the above-described embodiments, but should be defined only in accordance with the following claims and their equivalents.

1. An apparatus comprising:
a wound dressing defining a channel, the channel including a body portion having a width and a neck portion having a width less than the width of the body portion.

2. The apparatus of claim 1, further comprising:
a tube having a distal end portion, a width of the distal end portion of the tube substantially corresponding to the wide of the body portion of the channel
the tube being fluidically coupled to the wound dressing when the distal end portion is disposed within the body portion of the channel.

3. The apparatus of claim 1, wherein:
a portion of the wound dressing that the defines the neck portion of the channel is configured to flex when the distal end portion of the tube is moved through the neck portion of the wound dressing to or from the body portion of the channel of the wound dressing.

4. The apparatus of claim 1, further comprising:
a tube having a distal end portion configured to be disposed within the body portion of the channel, the tube being configured to substantially maintain its position within the body portion of the channel when suction is applied to the wound dressing via the tube.

5. The apparatus of claim 1, wherein the width of the neck portion is a first width of the neck portion, the neck portion having the first width when a tube is disposed within the body portion, the neck portion having a second width when a tube is passed through the neck portion to the body portion, the second width being greater than the first width.

6. The apparatus of claim 1, wherein the neck portion of the channel is flexible such that a tube can pass through the neck portion of the channel and into the body portion of the channel.

7. An apparatus, comprising:
a wound dressing defining a channel having a body portion, the body portion of the channel having a width; and
a tube configured to be disposed within the body portion of the channel,
the width of the body portion of the channel being substantially equal to a width of the tube, the body portion of the channel configured to movably secure the tube when disposed within the channel.

8. The apparatus of claim 7, wherein the wound dressing and the tube are collectively configured to substantially maintain a position of the tube within the body portion of the channel when suction is applied to the wound dressing via the tube.

9. The apparatus of claim 7, wherein the wound dressing movably secures the tube by a friction fit between the tube and the body portion of the channel.

10. The apparatus of claim 7, wherein the channel includes a neck portion having a width less than the width of the tube, the neck portion of the channel configured to maintain at least in part, a location of the tube within the body portion of the channel.

11. The apparatus of claim 7, wherein the channel includes a neck portion having a width less than the width of the tube, the neck portion being configured to flex when the distal end portion of the tube is moved through the neck portion of the channel to the body portion of the channel.

12. The apparatus of claim 7, wherein the body portion of the channel has a substantially rectangular cross-section.

13. The apparatus of claim 7, wherein the channel is a first channel, the tube is a first tube, the apparatus further comprising:
a second tube configured to be disposed within a second channel of the wound dressing,
the wound dressing and the first tube are configured to substantially maintain a position of the first tube within the first channel when suction is applied to the wound dressing via the first tube, the wound dressing and the second tube are configured to substantially maintain a position of the second tube within the second channel when a fluid is applied to the wound dressing via the second tube.

14. The apparatus of claim 7, wherein the channel is a first channel, the wound dressing further defining a second channel, the first channel being substantially perpendicular to the second channel.

15. The apparatus of claim 7, wherein the wound dressing includes a first surface configured to contact a wound of a patient and a second surface opposite the first surface, the second surface defining the channel.

16. The apparatus of claim 7, wherein the channel is a first channel, the wound dressing further defining a second channel, the first cannel intersecting the second channel at a substantially center point of a surface of the wound dressing.

17. The apparatus of claim 7, wherein the channel is a first channel, the wound dressing further defining a second channel, the first cannel intersecting the second channel at a substantially center point of a surface of the wound dressing such that the first channel and the second channel partition the wound dressing into four substantially equal quadrants, the wound dressing being monolithically formed.

18. The apparatus of claim 7, wherein the channel extends from a first side surface of the wound dressing to a second side surface of the wound dressing opposite the first side surface.

19. The apparatus of claim 7, wherein the wound dressing is monolithically formed.

20. The apparatus of claim 7, wherein the width of the body portion of the channel is less than a width of the wound dressing.

21. The apparatus of claim 7, wherein the wound dressing is substantially rectangular in shape.

22. A method, comprising:
disposing a wound dressing having a channel into contact with a wound of a patient;
disposing a first portion of a tube into the channel of the wound dressing such that the tube is fluidically coupled to the wound dressing;
connecting a second portion of the tube to a suction source; and
applying suction to the wound dressing via the tube.

23. The method of claim 22, further comprising:
removing the first portion of the tube from the channel without removing the dressing from contacting the wound of the patient.

24. The method of claim 22, wherein the disposing the first portion of the tube into the channel includes passing the first portion of the tube through a neck portion of the channel such that the tube is movably secured within a body portion of the channel.

25. The method of claim 22, wherein the channel is a first channel, the tube being a first tube, the method further comprising:
disposing a first portion of a second tube into a second channel of the wound dressing such that the second tube is fluidically coupled to the wound dressing; and
connecting a second portion of the second tube to an irrigation source.

26. The method of claim 22, further comprising:
disposing a semi-permeable sheet having a first surface and a second surface such that the first surface contacts the wound dressing, the wound dressing being fludically isolated from the second surface of the semi-permeable sheet.

27. An apparatus, comprising:
a wound dressing defining a channel having a body portion; and
a tube having a portion configured to be disposed within the body portion of the channel, the body portion of the channel having a width smaller than a width of the portion of the tube when the portion of the tube is outside of the channel, the body portion of the channel having a width equal to a width of the portion of the tube when the portion of the tube is disposed within the channel.

28. The apparatus of claim 27, wherein the wound dressing movably secures the tube by a friction fit between the tube and the body portion of the channel.

29. The apparatus of claim 27, wherein the wound dressing and the tube are collectively configured to substantially maintain a position of the tube within the body portion of the channel when suction is applied to the wound dressing via the tube.

30. The apparatus of claim 27, wherein the channel includes a neck portion having a width less than the width of the portion of the tube when the portion of the tube is outside of the channel, the neck portion being configured to flex when the portion of the tube is moved through the neck portion of the channel to the body portion of the channel.

31. An apparatus, comprising:
a wound dressing having a top surface, a bottom surface, a first side surface and a second side surface, the top surface defining a first channel and a second channel, the first channel being substantially perpendicular to the second channel, the first channel intersecting the second channel at a substantially center point on the top surface of the wound dressing, the first channel extending from the first side surface of the wound dressing to the second side surface of the wound dressing,
the bottom surface configured to contact a wound of a patient.
